(19) United States
(12) Patent Application Publication
(10) Pub. No.: US 2026/0338770 A1
(43) Pub. Date: Oct. 29, 2026

(54) DUAL DETECTION OF MAGNETIC AND DIELECTRIC SIGNALS FROM CORE/SHELL NANOPARTICLES

(71) Applicants: UNIVERSITY OF SOUTH FLORIDA, Tampa, FL (US); UNIVERSITY OF HOUSTON SYSTEM, Houston, TX (US)

(72) Inventors: Manh-Huong PHAN, Tampa, FL (US); Kee Young HWANG, Tampa, FL (US); T. Randall LEE, Houston, TX (US); Minh Dang NGUYEN, Houston, TX (US); Supawitch HOIJANG, Houston, TX (US); Dakota BROWN, Tampa, FL (US)

(21) Appl. No.: 19/660,142

(22) Filed: Apr. 27, 2026

Related U.S. Application Data
(60) Provisional application No. 63/794,921, filed on Apr. 25, 2025.

(51) Int. Cl. G01N 33/543; G01N 15/00; G01N 15/01; G01N 15/1031; G01N 33/575

(52) U.S. Cl. CPC G01N 33/54346; G01N 15/01; G01N 15/1031; G01N 33/575; G01N 2015/0038

(57) ABSTRACT

A magneto-LC resonance (MLCR) biosensing system and method for simultaneous detection of magnetic and dielectric signals from core/shell nanoparticles. The system comprises a coil of soft magnetic microwire wound into multiple turns defining an impedance sensing region, operated at a frequency above the coil's self-resonant frequency to induce a negative reactance. In this capacitive regime, the coil functions simultaneously as both a magnetic sensor and a dielectric sensor, measuring total impedance changes (ΔZtotal=ΔZm+ΔZd) as core/shell nanoparticles transit through the sensing region. A control system comprising a coil driver, transit driver, and impedance measurement circuitry acquires impedance data in real time.

### CROSS-REFERENCE TO RELATED APPLICATIONS

This application claims the benefit of U.S. Provisional Application No. 63/794,921, filed Apr. 25, 2025, the entire disclosure of which is incorporated herein by reference.

### STATEMENT REGARDING FEDERALLY SPONSORED RESEARCH OR DEVELOPMENT

This invention was made with government support under DE-FG02-07ER46438 awarded by the Department of Energy and AFOSR FA9550-23-1-0581 awarded by the Department of Air Force. The government has certain rights in the invention.

### BACKGROUND OF THE INVENTION

Biosensing platforms capable of detecting and characterizing disease markers, cellular interactions, and molecular binding events are of significant interest in medical diagnostics, environmental monitoring, and pharmaceutical development. Core/shell nanoparticles, such as Fe₃O₄@SiO₂ nanoparticles comprising a magnetic core and a biocompatible shell (e.g., a dielectric shell), have been widely explored as detection agents in such platforms. The magnetic core provides a magnetically responsive signal, while the shell offers biocompatibility and a feasible chemical surface for conjugation of biomolecules.

Existing biosensing modalities, including giant magnetoresistance (GMR) and giant magnetoimpedance (GMI) sensors, have been employed for detection of magnetic nanoparticles. However, these conventional sensors are generally limited to detecting either magnetic or dielectric signals, but not both simultaneously. Moreover, the detection sensitivity of such sensors is significantly reduced in the presence of non-magnetic dielectric shells.

### BRIEF SUMMARY OF THE INVENTION

In one aspect, the present disclosure provides a system comprising a coil comprising a soft magnetic microwire having a plurality of turns defining an impedance sensing region, and a control system comprising a coil driver electrically coupled to the coil to deliver an alternating current at a frequency sufficient to induce a negative reactance in the coil, a transit driver coupled to output a drive signal to cause a sample comprising core/shell nanoparticles to transit through the impedance sensing region, impedance measurement circuitry electrically coupled to the coil to measure impedance data while the core/shell nanoparticles transit through the impedance sensing region, and a data output to transmit the impedance data.

In another aspect, the present disclosure provides a method comprising operating a coil driver electrically coupled to a coil to deliver an alternating current at a frequency sufficient to induce a negative reactance in the coil, the coil comprising a soft magnetic microwire having a plurality of turns defining an impedance sensing region; operating a transit driver to output a drive signal to cause a sample comprising core/shell nanoparticles to transit through the impedance sensing region; operating impedance measurement circuitry electrically coupled to the coil to measure impedance data from the core/shell nanoparticles disposed within the sample; and transmitting the impedance data via a data output.

In a further aspect, the present disclosure provides a non-transitory computer-readable medium storing executable instructions to operate a coil driver electrically coupled to a coil to deliver an alternating current at a frequency sufficient to induce a negative reactance in the coil, operate a transit driver to output a drive signal to cause a sample comprising core/shell nanoparticles to transit through an impedance sensing region defined by the coil, operate impedance measurement circuitry electrically coupled to the coil to measure impedance data from the core/shell nanoparticles disposed within the sample, and transmit the impedance data via a data output.

### BRIEF DESCRIPTION OF THE DRAWINGS

FIG. 1A is a schematic diagram of a magneto-LC resonance (MLCR) biosensing system in a base configuration, in accordance with certain embodiments of the present disclosure.

FIG. 1B is a schematic diagram illustrating a coil equivalent-circuit model and impedance regime detail of the MLCR biosensing system, including a microfluidic cartridge embodiment, in accordance with certain embodiments of the present disclosure.

FIG. 1C is a schematic diagram of the MLCR biosensing system configured for intracellular nanoparticle detection, in accordance with certain embodiments of the present disclosure.

FIG. 1D is a schematic diagram of the MLCR biosensing system configured with an energy modulation subsystem and a dual-reservoir sample introduction pathway, in accordance with certain embodiments of the present disclosure.

FIG. 2 is a schematic diagram of a networked cloud-enabled data analysis architecture for the MLCR biosensing system, in accordance with certain embodiments of the present disclosure.

FIGS. 3A and 3B are transmission electron microscopy (TEM) images and X-ray diffraction (XRD) patterns of core Fe₃O₄(192 nm) and core/shell Fe₃O₄(192 nm)@SiO₂(30 nm) nanoparticles, as described in the Working Examples section herein.

FIGS. 4A through 4C are graphs, X-ray diffraction (XRD) patterns, and transmission electron microscopy (TEM) images illustrating SiO₂ shell thickness as a function of Fe₃O₄ content, phase characterization, and morphology of Fe₃O₄(339 nm)@SiO₂ nanoparticles with shell thicknesses of 17, 25, 30, and 48 nm, as described in the Working Examples section herein.

FIGS. 5A through 5D are graphs illustrating magnetization versus magnetic field (M-H) curves for core Fe₃O₄(192 nm) nanoparticles and core/shell Fe₃O₄(192 nm)@SiO₂(30 nm) nanoparticles at magnetic core masses of 8.5 mg and 14.0 mg, as described in the Working Examples section herein.

FIGS. 6A through 6D are graphs illustrating magnetization versus magnetic field (M-H) curves for core Fe₃O₄(339 nm) nanoparticles and core/shell Fe₃O₄(339 nm)@SiO₂(30 nm) nanoparticles at magnetic core masses of 4.0 mg and 11.2 mg, as described in the Working Examples section herein.

FIGS. 7A through 7F are graphs illustrating magnetization versus magnetic field (M-H) curves in emu/g for core Fe₃O₄(339 nm) nanoparticles and core/shell Fe₃O₄(339 nm)@SiO₂ nanoparticles with shell thicknesses of 17, 25, 30, and 48 nm, and saturation magnetization as a function of SiO₂ shell thickness, as described in the Working Examples section herein.

FIGS. 8A through 8D are graphs illustrating changes in total impedance as a function of time for core Fe₃O₄(192 nm) nanoparticles and core/shell Fe₃O₄(192 nm)@SiO₂(30 nm) nanoparticles at magnetic core masses of 8.5 mg and 14.0 mg flowing through the MLCR sensor, as described in the Working Examples section herein.

FIGS. 9A and 9B are graphs illustrating comparative detection sensitivity of core nanoparticles and core/shell nanoparticles, including Fe₃O₄(192 nm)@SiO₂(30 nm) and Fe₃O₄(339 nm)@SiO₂(30 nm) nanoparticles, as a function of magnetic core mass, as described in the Working Examples section herein.

FIGS. 10A and 10B are graphs illustrating comparative detection sensitivity for core/shell Fe₃O₄(192 nm)@SiO₂(30 nm) and Fe₃O₄(339 nm)@SiO₂(30 nm) nanoparticles as a function of core/shell mass, and the linear relationship between detection sensitivity and core/shell mass, as described in the Working Examples section herein.

FIGS. 11A and 11B are graphs illustrating detection sensitivity of Fe₃O₄(339 nm)@SiO₂ nanoparticles as a function of SiO₂ shell thickness at magnetic core masses of 4.0 mg and 14.0 mg, as described in the Working Examples section herein.

### DETAILED DESCRIPTION OF EMBODIMENTS

The present disclosure relates to magneto-LC resonance (MLCR) biosensing systems and methods configured for simultaneous detection of magnetic and dielectric signals from core/shell nanoparticles. The present inventors have discovered that by operating a coil comprising a soft magnetic microwire at a frequency above the self-resonant frequency of the coil—thereby placing the coil in a capacitive (negative reactance) regime—the coil functions simultaneously as both a magnetic sensor and a dielectric sensor within a single impedance measurement. This dual-signal detection capability enables the system to measure total impedance changes expressed as ΔZₜₒₜₐₗ=ΔZₘ+ΔZ_{d}, where ΔZₘ reflects the magnetic impedance contribution and ΔZ_{d} reflects the dielectric impedance contribution, as nanoparticle-associated samples flow through an impedance sensing region defined by the coil.

Conventional biosensing modalities, such as GMR and GMI sensors, are generally limited to detecting either magnetic or dielectric signals, and their detection sensitivity is significantly reduced in the presence of non-magnetic dielectric shells. In contrast, the MLCR biosensing systems and methods disclosed herein preserve or enhance detection sensitivity for core/shell nanoparticles, including Fe₃O₄@SiO₂ nanoparticles, by leveraging both the magnetic properties of the core and the dielectric properties of the shell. The present disclosure demonstrates that the MLCR sensor achieves a remarkable enhancement in detection sensitivity relative to a GMI sensor under identical testing conditions. By tuning the thickness of the dielectric shell, the MLCR sensor can detect subtle changes in dielectric properties, opening avenues for real-time monitoring of biological variations associated with disease progression and other analyte characteristics.

The following detailed description sets forth various embodiments of the present disclosure with reference to the accompanying drawings. It is to be understood that the described embodiments are illustrative and not limiting, and that various modifications, substitutions, and alterations may be made without departing from the spirit and scope of the present disclosure as defined by the appended claims. Reference numerals are used consistently throughout the figures and description to refer to like elements. Where a range of values is provided, each intervening value between the upper and lower limit of that range is also specifically disclosed.

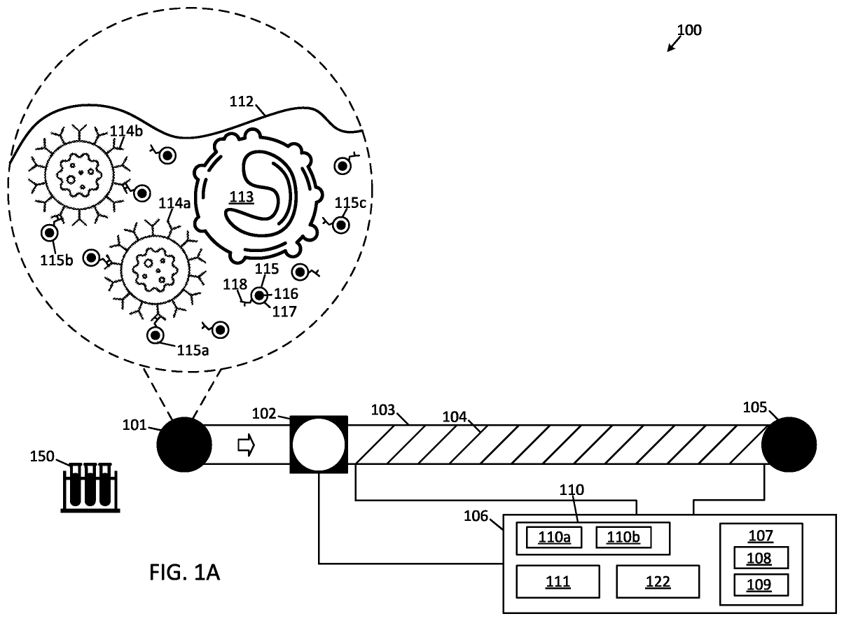

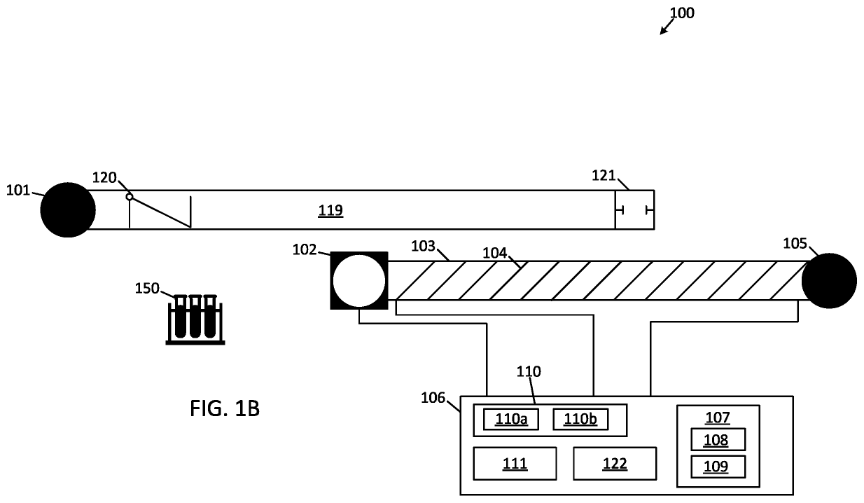

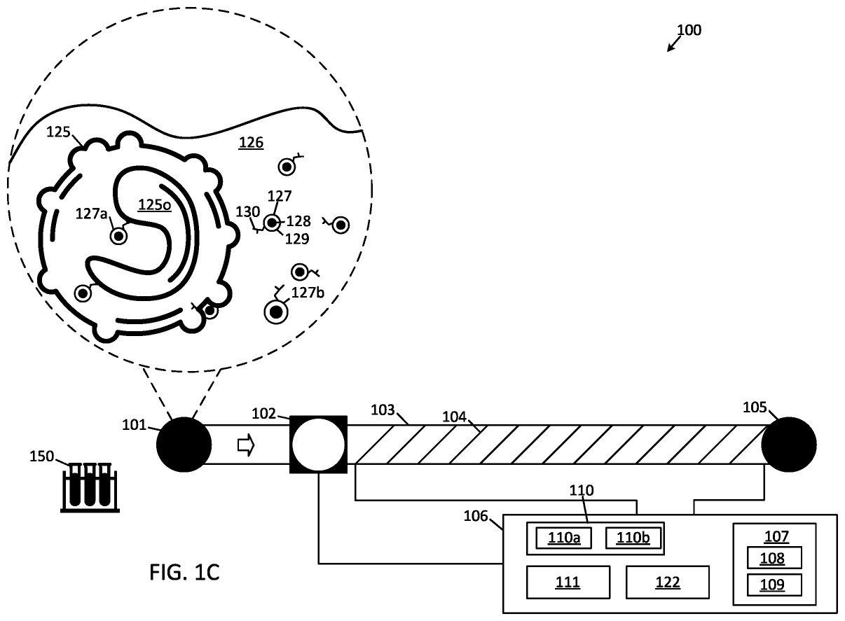

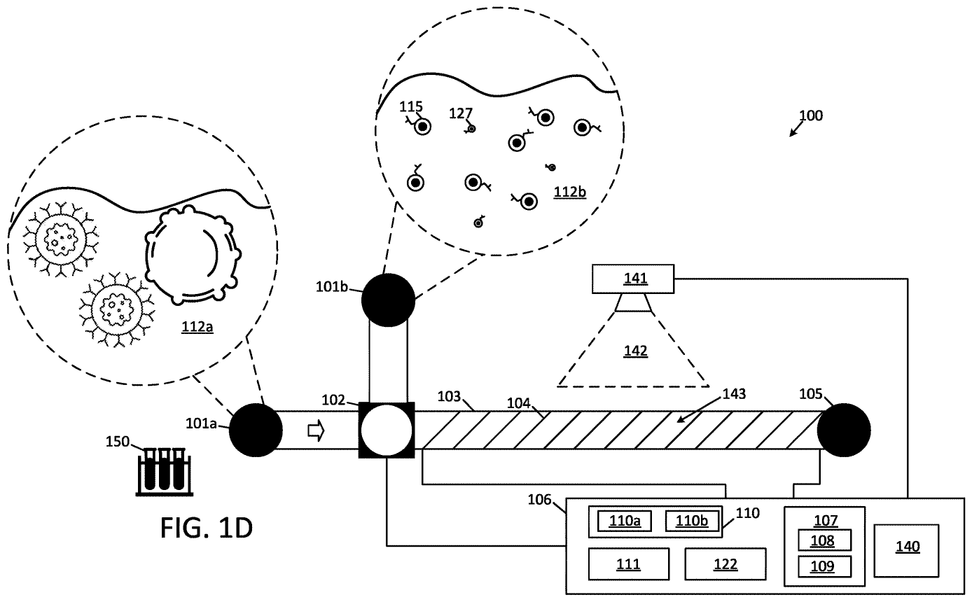

Turning now to FIG. 1A, there is depicted a magneto-LC resonance (MLCR) biosensing system 100 configured to detect both magnetic and dielectric signals from core/shell nanoparticles transiting through an impedance sensing coil. System 100 measures total impedance changes—expressed as ΔZₜₒₜₐₗ=ΔZₘ+ΔZ_{d}, where ΔZₘ reflects the magnetic impedance contribution and ΔZ_{d} reflects the dielectric impedance contribution—as nanoparticle-associated samples flow through an impedance sensing region. This dual-signal detection enables simultaneous, real-time characterization of the magnetic and dielectric properties of nanoparticles and bound analytes within a single measurement pass.

In the embodiment of FIG. 1A, a prepared sample 112 is introduced into system 100 via sample receptacle 101 and is propelled by actuator/pump 102 through conduit 103, which passes through the interior of coil 104. After transit through the impedance sensing region, the sample is collected at receiving receptacle or drain 105. Conduit 103 is sized to permit transit of the sample including any cells or particles-through the interior of coil 104. In embodiments in which sample 112 contains cells, conduit 103 may have an inner diameter of about 0.5 mm to about 10 mm. In one embodiment, conduit 103 comprises a plastic tube having an inner diameter of about 4 mm and a length of about 7 mm. In some embodiments, conduit 103 is implemented as a removable microfluidic cartridge 119, which may be replaced between sample runs to prevent cross-contamination and to accommodate different sample types. Sample volume may range from about 0.001 mL to about 50 mL. Actuator/pump 102 may be pressure-driven, peristaltic, electroosmotic, centrifugal, or gravity-driven.

Coil 104 serves as the primary impedance sensing element of system 100. In one embodiment, coil 104 comprises a soft magnetic microwire wound into multiple turns around conduit 103—for example, an ultrasoft magnetic Co—Fe—Si—B—Nb microwire of about 60 μm diameter wound into about 15 turns. The soft magnetic character of the microwire renders coil 104 sensitive to small magnetic fields produced by magnetic nanoparticles transiting the coil interior, contributing the magnetic impedance change ΔZₘ. Parasitic inter-turn capacitance of coil 104 additionally renders the coil sensitive to the dielectric properties of material within the coil interior, contributing the dielectric impedance change ΔZ_{d}. System 100 is driven at a frequency above the self-resonant frequency of coil 104, placing coil 104 in a capacitive (negative reactance) regime. In this regime, total coil impedance is dominated by the capacitive term and is inversely proportional to parasitic capacitance; changes in the dielectric constant of material inside the coil alter capacitance and thereby alter total coil impedance, enabling coil 104 to function simultaneously as both a magnetic sensor and a dielectric sensor within a single impedance measurement. The total impedance change observed as nanoparticles transit coil 104 is characterized by ΔZₜₒₜₐₗ=ΔZₘ+ΔZ_{d}, capturing both contributions.

Coil control circuitry 110 comprises coil driver 110a, which delivers AC excitation to coil 104 at a selected operating frequency, and impedance measurement circuitry 110b, which measures total coil impedance in real time as nanoparticles transit through the sensing region. The operating frequency of system 100 may range from about 100 kHz to about 1 GHz, and is selected based on the self-resonant frequency of the particular coil 104 employed so as to maintain operation in the capacitive regime. Coil 104 may comprise about 1 to about 1,000 turns; microwire diameter may range from about 1 μm to about 500 μm. Impedance measurement circuitry 110b may be implemented as a vector network analyzer (VNA), lock-in amplifier, or impedance analyzer integrated circuit.

Control system 106 orchestrates the operation of coil driver 110a, impedance measurement circuitry 110b, and sample transit via transit driver 111. Control system 106 receives impedance data from impedance measurement circuitry 110b and transmits impedance data via data output 122 for downstream analysis or display. In some embodiments, control system 106 includes computing system 107, comprising processor 108 and memory 109, configured to process impedance data and to resolve individual magnetic (ΔZₘ) and dielectric (ΔZ_{d}) impedance components. Memory 109 is a non-transitory computer-readable memory storing instructions that, when executed by processor 108, cause the processor to: resolve, from the impedance data, a magnetic impedance component and a dielectric impedance component; and determine a presence or concentration of a target analyte based on the magnetic impedance component and the dielectric impedance component.

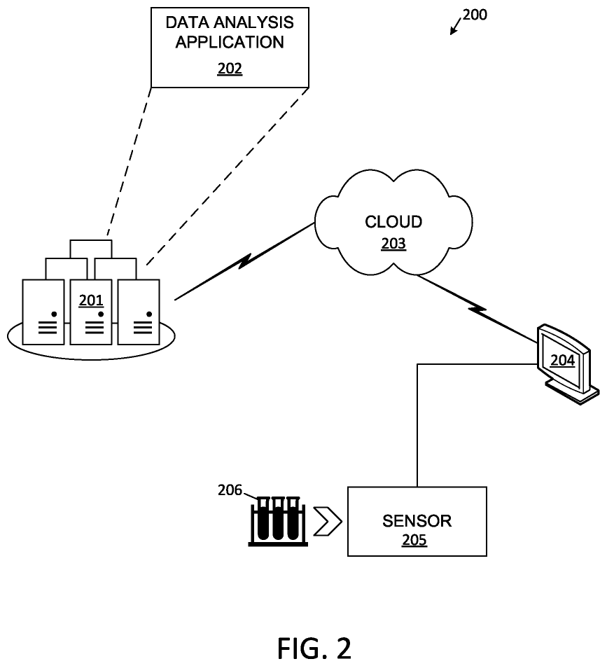

In some embodiments, memory 109 further stores instructions that, when executed by processor 108, cause the processor to: detect, from the impedance data, a temporal change in the magnetic impedance component or the dielectric impedance component as the core/shell nanoparticles transit through the impedance sensing region; and determine binding of target molecules to the core/shell nanoparticles based on the temporal change. In some embodiments, the instructions stored in memory 109 are stored on a non-transitory computer-readable medium separate from or integrated with computing system 107. Such a non-transitory computer-readable medium stores executable instructions to: operate coil driver 110a electrically coupled to coil 104 to deliver an alternating current at a frequency sufficient to induce a negative reactance in coil 104; operate transit driver 111 to output a drive signal to cause a sample comprising core/shell nanoparticles to transit through the impedance sensing region; operate impedance measurement circuitry 110b electrically coupled to coil 104 to measure impedance data from the core/shell nanoparticles disposed within the sample; and transmit the impedance data via data output 122. In some embodiments, the instructions further cause the processor to resolve, from the impedance data, a magnetic impedance component and a dielectric impedance component, and to determine a presence or concentration of a target analyte based on the magnetic impedance component and the dielectric impedance component. Control system 106 may be implemented as a programmable logic controller (PLC), field-programmable gate array (FPGA), single-board computer, application-specific integrated circuit (ASIC), or general-purpose computer executing stored software. Data analysis may be performed locally within system 100 or offloaded to remote or cloud-based infrastructure, as further described in connection with FIG. 2.

System 100 is configured to detect target analytes in a sample that may also contain non-target material. In one embodiment, the target analytes comprise cancer cells 114a and 114b, and non-target material comprises healthy cells 113. Core/shell nanoparticles 115 serve as detection agents. In some embodiments, nanoparticles 115 comprise Fe₃O₄@SiO₂ particles having magnetic cores 116 and dielectric shells 117. Exemplary Fe₃O₄ core diameters include about 192 nm and about 339 nm; exemplary SiO₂ shell thicknesses include about 17 nm, about 25 nm, about 30 nm, and about 48 nm. In some embodiments, a ratio of the diameter of magnetic cores 116 to the thickness of dielectric shells 117 is from about 4:1 to about 30:1. For example, a core diameter of about 192 nm with a shell thickness of about 48 nm yields a ratio of about 4:1, while a core diameter of about 339 nm with a shell thickness of about 17 nm yields a ratio of about 20:1; other ratios within the 4:1 to 30:1 range are achievable by appropriate selection of core diameter and shell thickness. Core diameter and shell thickness may be adjusted for a particular application.

In some embodiments, the magnetic core material comprises a superparamagnetic iron oxide or other superparamagnetic composition, such as a superparamagnetic ferrite, having a crystallite size of less than about 15 nanometers; Fe₃O₄ nanocrystallites with crystallite sizes of approximately 9 to 12 nm have been demonstrated. In some embodiments, dielectric shells 117 comprise a non-magnetic dielectric material having a relative permittivity of at least 2.0 at room temperature; SiO₂ shells exhibit a relative permittivity in the range of approximately 3.9 to 4.0 for bulk material, and other suitable dielectric shell materials include TiO₂, Al₂O₃, ZrO₂, biocompatible polymers, and lipid coatings, each having a relative permittivity of at least 2.0 at room temperature. In some embodiments, nanoparticles 115 are functionalized with binding moiety 118, which selectively binds to a surface marker of the target analyte. Binding moiety 118 may comprise an antibody, aptamer, peptide, small molecule, or other selective binding agent.

In some embodiments, the core/shell architecture is inverted relative to the Fe₃O₄@SiO₂ configuration described above. In such embodiments, core/shell nanoparticles comprise dielectric cores and magnetic shells encapsulating or at least partially coating the dielectric cores. For example, a silica core may be coated with a magnetic iron oxide shell or other magnetically responsive material. Such inverted core/shell nanoparticles are likewise capable of transiting through the impedance sensing region defined by coil 104, producing detectable combined magnetic and dielectric impedance changes as they do so. The dielectric cores contribute a dielectric impedance change (ΔZ_{d}) and the magnetic shells contribute a magnetic impedance change (ΔZₘ). The selection of core/shell architecture—magnetic core with dielectric shell, or dielectric core with magnetic shell—may be made based on the target application, desired surface chemistry for functionalization, biocompatibility requirements, or other factors known to one of ordinary skill in the art. Receptacle 150 (and, in the FIG. 1D embodiment, receptacle 101b) stores the core/shell nanoparticles prior to introduction into the sensing system.

As core/shell nanoparticles 115 transit through the impedance sensing region defined by coil 104, impedance measurement circuitry 110b measures impedance data as a function of time. The passage of nanoparticles through the sensing region produces a temporal change in the magnetic impedance component (ΔZₘ) and/or the dielectric impedance component (ΔZ_{d}) of the total coil impedance. In some embodiments, computing system 107 is configured to detect, from the impedance data, such a temporal change in the magnetic impedance component or the dielectric impedance component as the core/shell nanoparticles transit through the impedance sensing region, and to determine binding of target molecules to the core/shell nanoparticles based on the temporal change. For example, core/shell nanoparticles 115 functionalized with binding moiety 118 that have bound to target cancer cells 114a or 114b produce a temporal impedance profile—characterized by the time-dependent evolution of ΔZₘ and ΔZ_{d} during transit—that is distinguishable from the profile produced by unbound nanoparticles 115c, enabling processor 108, executing instructions stored in memory 109, to determine that binding of target molecules to the core/shell nanoparticles has occurred. The transit of core/shell nanoparticles through the sensing region generates a characteristic time-varying impedance signal from which binding events and analyte characteristics are resolved.

In some embodiments, a method of operating system 100 comprises: operating coil driver 110a, which is electrically coupled to coil 104, to deliver an alternating current at a frequency sufficient to induce a negative reactance in coil 104, coil 104 comprising a soft magnetic microwire having a plurality of turns defining an impedance sensing region; operating transit driver 111 to output a drive signal to actuator/pump 102 to cause a sample comprising core/shell nanoparticles to transit through the impedance sensing region; operating impedance measurement circuitry 110b, which is electrically coupled to coil 104, to measure impedance data from the core/shell nanoparticles disposed within the sample; and transmitting the impedance data via data output 122. In some embodiments, the method further comprises providing sample conduit 103 disposed within the impedance sensing region and carrying the sample through the impedance sensing region via conduit 103. In some embodiments, the method further comprises resolving, from the impedance data, a magnetic impedance component (ΔZₘ) and a dielectric impedance component (ΔZ_{d}); and determining a presence or concentration of a target analyte based on the magnetic impedance component and the dielectric impedance component. In some embodiments, the method further comprises detecting, from the impedance data, a temporal change in the magnetic impedance component or the dielectric impedance component as the core/shell nanoparticles transit through the impedance sensing region; and determining binding of target molecules to the core/shell nanoparticles based on the temporal change. The foregoing method steps are non-limiting examples of how system 100 may be operated and are not intended to limit the scope of the claims.

System 100 as depicted in FIG. 1A supports multiple detection applications. In one embodiment directed to cancer cell detection, nanoparticles 115 functionalized with binding moiety 118 specific to a cancer cell surface marker are combined with a biological sample such as blood, a cell suspension, or a processed biopsy specimen and introduced via receptacle 101 to flow through conduit 103 and coil 104. Nanoparticle-labeled cancer cells 114a, 114b exhibit an impedance signature—comprising both a magnetic component (ΔZₘ) from the Fe₃O₄ cores and a dielectric component (ΔZ_{d}) reflecting the SiO₂ shell and cell membrane—that is distinct from unlabeled non-target cells 113 or unbound nanoparticles 115c. Impedance measurement circuitry 110b captures this signature in real time as the sample transits the sensing region, and computing system 107 processes the data to identify and enumerate cancer cells; detecting cancer cells 114a and 114b based on the temporal change in the magnetic or dielectric impedance component during transit is one example of determining binding of target molecules to the core/shell nanoparticles based on the temporal change. In a further embodiment, system 100 supports live-cell metabolic and viability monitoring by detecting dielectric impedance shifts (ΔZ_{d}) characteristic of metabolically active versus metabolically inactive cells transiting coil 104. In an additional embodiment, system 100 is used to characterize particles in industrial or environmental fluid samples, leveraging the combined magnetic/dielectric impedance signature to classify particles by composition and surface state.

Turning now to FIG. 1B, there is illustrated the non-ideal equivalent circuit model of coil 104, which underlies the dual magnetic and dielectric detection capability of system 100, and also depicts the microfluidic cartridge embodiment of the sample conduit. The equivalent circuit model of coil 104 comprises an inductance (L) representing the coil's inductive behavior, a series resistance (Rz) representing resistive losses of the microwire, and a parasitic capacitance (CL) in parallel representing inter-turn capacitance inherent to the wound microwire geometry. At frequencies below the self-resonant frequency of coil 104, the coil exhibits inductive (positive reactance) behavior. At frequencies above the self-resonant frequency—the operating regime selected for system 100—the coil operates in a capacitive (negative reactance) regime, wherein total coil impedance is dominated by the capacitive term and is inversely proportional to CL. Because CL is sensitive to the dielectric constant of material within and around conduit 103, changes in the dielectric properties of nanoparticle-laden samples transiting the coil interior produce measurable dielectric impedance changes (ΔZ_{d}). Simultaneously, the magnetic permeability of the soft magnetic microwire responds to the magnetic fields of transiting nanoparticles, producing the magnetic impedance contribution (ΔZₘ). The total measured impedance change is thus ΔZₜₒₜₐₗ=ΔZₘ+ΔZ_{d}. Coil driver 110a is configured to drive coil 104 at the selected operating frequency to maintain operation above the self-resonant frequency in the capacitive regime. Impedance measurement circuitry 110b measures total coil impedance in real time.

As further depicted in FIG. 1B, microfluidic cartridge 119 is removably receivable in a cartridge receiver of system 100. As shown, cartridge 119 slides into and seats within the region defined by conduit 103 within coil 104, such that the sample channel of cartridge 119 aligns with the impedance sensing region. Cartridge 119 includes first stop valve 120, which regulates sample flow within the cartridge, and drain 121, which couples with receiving receptacle 105 upon insertion of the cartridge into the cartridge receiver. In some embodiments, the cartridge receiver is a structural element of system 100 configured to removably receive and align cartridge 119 within the impedance sensing region defined by coil 104. The removable nature of cartridge 119 facilitates replacement between sample runs to prevent cross-contamination and to accommodate different sample types or analytes.

In some embodiments, a method of operating system 100 in the configuration of FIG. 1B comprises operating coil driver 110a to deliver an alternating current at a frequency sufficient to induce a negative reactance in coil 104, wherein the frequency is selected to place coil 104 in the capacitive regime above its self-resonant frequency; operating transit driver 111 to output a drive signal causing the sample comprising core/shell nanoparticles to transit through the impedance sensing region defined by coil 104; operating impedance measurement circuitry 110b to measure impedance data from the core/shell nanoparticles disposed within the sample; and transmitting the impedance data via data output 122. In some embodiments, the method further comprises providing microfluidic cartridge 119 as the sample conduit disposed within the impedance sensing region, inserting cartridge 119 into the cartridge receiver of system 100, and carrying the sample through the impedance sensing region via cartridge 119. The coil equivalent-circuit model depicted in FIG. 1B provides the basis for operating frequency selection for coil driver 110a across all embodiments of system 100. By selecting an operating frequency that places coil 104 in the capacitive (negative reactance) regime above its self-resonant frequency, system 100 achieves simultaneous sensitivity to both the magnetic (ΔZₘ) and dielectric (ΔZ_{d}) properties of transiting nanoparticle-sample combinations. This principle applies across all detection contexts described in connection with FIG. 1A, FIG. 1C, and FIG. 1D.

Turning now to FIG. 1C, there is depicted an embodiment of system 100 configured for intracellular detection. System 100 elements shared with FIG. 1A including sample receptacle 101, actuator/pump 102, conduit 103, coil 104, receiving receptacle or drain 105, control system 106, computing system 107, processor 108, memory 109, coil control circuitry 110, coil driver 110a, impedance measurement circuitry 110b, transit driver 111, sample 112, non-target cells 113, and data output 122—are configured and operable as described in connection with FIG. 1A, and that description is incorporated herein by reference. In the intracellular embodiment of FIG. 1C, cells 125 are present in the sample and have been loaded with core/shell nanoparticles 127. Nanoparticles 127 are sized and configured for cellular internalization—for example, via endocytosis or other cellular uptake mechanisms. Each nanoparticle 127 comprises a magnetic core 128 and a dielectric shell 129. Nanoparticle 127 is functionalized with binding moiety 130, which is configured to bind to an intracellular target such as an organelle 125o—for example, mitochondria, lysosomes, or endosomes. After internalization and intracellular trafficking, nanoparticles 127 reside within cells 125 in proximity to or bound to target organelle 125o. As nanoparticle-loaded cells 125 transit through coil 104, impedance measurement circuitry 110b detects the combined magnetic (ΔZₘ) and dielectric (ΔZ_{d}) impedance signature of internalized nanoparticles 127, enabling characterization of intracellular targets at single-cell resolution. Core 128 may comprise a superparamagnetic iron oxide or other superparamagnetic composition. Shell 129 may comprise a biocompatible dielectric material compatible with intracellular environments, such as silica, alumina, zirconia, a biocompatible polymer, or a lipid-based coating. Binding moiety 130 may comprise an organelle-targeting peptide, antibody fragment, aptamer, or other selective binding agent.

In some embodiments, a method of operating system 100 in the intracellular configuration of FIG. 1C comprises: operating coil driver 110a, electrically coupled to coil 104 comprising a soft magnetic microwire having a plurality of turns defining an impedance sensing region, to deliver an alternating current at a frequency sufficient to induce a negative reactance in coil 104; operating transit driver 111 to output a drive signal causing a sample comprising core/shell nanoparticles—specifically, cells 125 that have internalized smaller core/shell nanoparticles 127—to transit through the impedance sensing region; operating impedance measurement circuitry 110b to measure impedance data from the core/shell nanoparticles disposed within the sample; and transmitting the impedance data via data output 122. In some embodiments, the method further comprises resolving, from the impedance data, a magnetic impedance component and a dielectric impedance component; and determining a presence or concentration of a target analyte—such as organelle 125o or another intracellular structure—based on the magnetic impedance component and the dielectric impedance component. In some embodiments, the method further comprises detecting, from the impedance data, a temporal change in the magnetic impedance component or the dielectric impedance component as the internalized core/shell nanoparticles transit through the impedance sensing region within host cells 125; and determining binding of target molecules to the core/shell nanoparticles based on the temporal change—for example, determining that nanoparticles 127 have bound to intracellular organelles 125o.

The intracellular embodiment of FIG. 1C enables detection and characterization of intracellular targets that are inaccessible to surface-binding nanoparticles. In one embodiment directed to organelle function monitoring, nanoparticles 127 functionalized with a mitochondria-targeting peptide as binding moiety 130 are delivered to cells 125 in culture. Cells 125 that have internalized nanoparticles 127 bound to mitochondria (organelle 1250) exhibit a combined magnetic/dielectric impedance signature upon transit through coil 104, reflecting the intracellular distribution and local dielectric environment of the targeted organelle. This enables monitoring of mitochondrial structural integrity and metabolic state at single-cell resolution-applications relevant to toxicology screening, drug efficacy assessment, and disease characterization. In another embodiment, nanoparticles 127 are directed to lysosomes via pH-sensitive or receptor-targeted binding moieties 130 to probe lysosomal activity, a marker relevant in cancer biology and lysosomal storage diseases.

Turning now to FIG. 1D, there is depicted an embodiment of system 100 further comprising an energy delivery subsystem and a dual-reservoir sample introduction pathway. System 100 elements shared with FIG. 1A—including coil 104, coil control circuitry 110, coil driver 110a, impedance measurement circuitry 110b, conduit 103, receiving receptacle or drain 105, control system 106, computing system 107, processor 108, memory 109, transit driver 111, data output 122, non-target cells 113, target cancer cells 114a and 114b, core/shell nanoparticles 115, binding moiety 118, and microfluidic cartridge 119—are configured and operable as described in connection with FIG. 1A, and that description is incorporated herein by reference. In the embodiment of FIG. 1D, the sample introduction pathway comprises a first receptacle 101a, which stores a biological or analyte-containing sample 112a (e.g., a cell suspension, plasma, or other biological fluid), and a second receptacle 101b, which stores a suspension of core/shell nanoparticles 115. Actuator/pump 102 draws from both receptacles 101a and 101b and combines their contents to form combined sample 112b, which is then driven through conduit 103 and coil 104. This in-line mixing configuration provides controlled nanoparticle-to-sample ratios immediately prior to sensing, reducing pre-incubation requirements and enabling rapid assay turnaround. In alternative embodiments, a single receptacle 101 may be used with a sample that has been pre-mixed with nanoparticles 115 prior to introduction.

As further depicted in FIG. 1D, heater driver 140 controls heater 141 to deliver energy 142 to a heated region 143 that coincides with or is adjacent to the impedance sensing region defined by coil 104. Energy delivery by heater 141 modulates the dielectric properties of the sample material within the sensing region, altering the dielectric impedance contribution (ΔZ_{d}) in a manner that enhances discrimination between different analyte states. In some embodiments, computing system 107 comprises a processor 108 and a non-transitory computer-readable memory 109 storing instructions that, when executed by processor 108, cause the processor to: resolve, from the impedance data, a magnetic impedance component and a dielectric impedance component; and determine a characteristic of a target analyte based on a change in the dielectric impedance component induced by energy delivered by heater 141. For example, processor 108 may determine whether a cell is cancerous or healthy, or whether a molecular target is in a first or second structural or binding state, by measuring the change in ΔZ_{d} that occurs when heater 141 delivers energy 142 to the sample while the sample transits the sensing region; cells or molecules in different states respond differently to the applied energy, producing distinguishable changes in ΔZ_{d}. Energy may be delivered by heater 141 as resistive heating, laser or infrared irradiation, ultrasound, microwave energy, or an AC magnetic field applied at a frequency separable from the operating frequency of coil driver 110a to permit independent acquisition of the impedance measurement signal. The temperature increase induced in heated region 143 may range from about 0.1 °C to about 15 °C; heater power may range from about 0.001 W to about 100 W. Control system 106 coordinates the timing and magnitude of energy delivery by heater driver 140 with the impedance measurement cycle of impedance measurement circuitry 110b. In some embodiments in which an AC magnetic field is used as the energy source delivered by heater 141, the frequency of the applied AC magnetic field is distinct from the operating frequency of coil driver 110a, permitting the impedance measurement signal from impedance measurement circuitry 110b to be acquired independently of the energy delivery signal.

In some embodiments, a method of operating system 100 in the energy modulation configuration of FIG. 1D comprises: operating coil driver 110a, electrically coupled to coil 104 comprising a soft magnetic microwire having a plurality of turns defining an impedance sensing region, to deliver an alternating current at a frequency sufficient to induce a negative reactance in coil 104; operating transit driver 111 to output a drive signal causing a sample comprising core/shell nanoparticles—specifically, combined sample 112b comprising core/shell nanoparticles 115 and, optionally, smaller core/shell nanoparticles 127—to transit through the impedance sensing region; operating impedance measurement circuitry 110b to measure impedance data from the core/shell nanoparticles disposed within the sample; operating heater 141, via heater driver 140, to deliver energy 142 to the sample within the impedance sensing region while the core/shell nanoparticles transit through the impedance sensing region; and transmitting the impedance data via data output 122. In some embodiments, the method further comprises resolving, from the impedance data, a magnetic impedance component and a dielectric impedance component; and determining a characteristic of a target analyte based on a change in the dielectric impedance component induced by the energy delivered by heater 141. The characteristic may be, for example, cell type (e.g., cancerous versus healthy), molecular binding state, protein conformation, or cell viability, any of which may produce a distinguishable change in ΔZ_{d} when the sample is subjected to the energy delivered by heater 141 during transit through the sensing region.

The energy modulation embodiment of FIG. 1D is suited to applications that benefit from amplified discrimination between analyte states. In one embodiment directed to biomarker state discrimination, nanoparticles 115 functionalized with binding moiety 118 specific to a target analyte—such as a protein in a particular structural or binding state—are combined with a biological sample (e.g., plasma or cerebrospinal fluid). As nanoparticle-analyte complexes transit coil 104, heater 141 delivers energy 142 to heated region 143, differentially stimulating distinct analyte states to elicit distinguishable dielectric impedance responses (ΔZ_{d}), thereby enabling determination of a characteristic of the target analyte based on the energy-induced change in the dielectric impedance component. In another embodiment directed to metabolic state discrimination, energy from heater 141 enhances dielectric contrast between metabolically active and metabolically inactive cell populations transiting coil 104, enabling more precise enumeration and classification. In a further embodiment, the dual-reservoir configuration of receptacles 101a and 101b enables just-in-time mixing of nanoparticles 115 with biological sample 112a, improving assay reproducibility and extending nanoparticle stability.

Turning now to FIG. 2, there is depicted a networked platform 200 that extends the capabilities of the sensing system described in connection with FIG. 1A through FIG. 1D by providing distributed, cloud-enabled data acquisition and analysis. Platform 200 comprises one or more servers 201 executing a data analysis application 202. Servers 201 are communicatively coupled via cloud network 203 to a user-facing computer 204. Impedance sensor 205, which corresponds in function and structure to system 100 as described in connection with FIG. 1A, acquires impedance data from sample with particles 206 as the particles transit the impedance sensing region. Impedance sensor 205 transmits the acquired impedance data via cloud network 203 to server(s) 201, where data analysis application 202 performs processing operations including resolving magnetic and dielectric impedance components (ΔZₘ and ΔZ_{d}), identifying and quantifying target analytes, and generating analytical reports or diagnostic outputs. Results are delivered from server(s) 201 via cloud network 203 to computer 204 for presentation to a clinician, researcher, or systems operator. In some embodiments, multiple impedance sensors 205 are networked to a common set of server(s) 201 and data analysis application 202, enabling centralized, scalable processing of impedance data from a plurality of geographically distributed sensing locations.

In some embodiments, a method of operating system 100 in the networked architecture of FIG. 2 comprises transmitting the impedance data via data output 122 of the sensing system—corresponding to impedance sensor 205—to a remote computing resource, such as server(s) 201, via cloud network 203. In some embodiments, the method further comprises resolving, from the impedance data transmitted to server(s) 201 and processed by data analysis application 202, a magnetic impedance component and a dielectric impedance component; and determining a presence or concentration of a target analyte based on the magnetic impedance component and the dielectric impedance component. Data output 122 of impedance sensor 205 may be implemented as a wired interface, a wireless interface, or an application programming interface (API) configured to transmit impedance data to a remote or cloud-based computing resource. The networked architecture of platform 200 depicted in FIG. 2 enables a variety of distributed and resource-efficient operational contexts. In a clinical deployment embodiment, impedance sensor 205 is located at a point-of-care site or clinical laboratory, while data analysis application 202 running on server(s) 201 performs cloud-based analysis and returns diagnostic results to a clinician's computer 204. This architecture allows resource-constrained sites to benefit from sophisticated analytical algorithms without local computational infrastructure. In a multi-site environmental monitoring embodiment, multiple impedance sensors 205 are deployed at geographically distributed locations—such as water treatment facilities or industrial monitoring points—and cloud network 203 aggregates impedance data for centralized analysis by data analysis application 202. In a translational research embodiment, data analysis application 202 applies machine learning or statistical algorithms to longitudinal impedance datasets aggregated from multiple sensors 205 and sites, enabling discovery of novel combined magnetic/dielectric signatures and facilitating model refinement as new data are acquired.

The following examples are provided to illustrate certain embodiments of the present disclosure and are not intended to limit the scope of the claims. The materials, methods, and conditions set forth below are exemplary and may be varied by one of ordinary skill in the art without departing from the spirit and scope of the present disclosure. Unless otherwise indicated, all parts and percentages are by weight, all temperatures are in degrees Celsius, and all measurements were performed under ambient conditions.

The following first considers the working principle of a conductive magnetic coil, starting with Faraday's Law of Electromagnetic Induction:

$$∮ \vec{E} \cdot \vec{d s} = - \frac{d}{dt} \int \int \vec{B} \cdot \vec{d a} \qquad ( 1 )$$

where \vec{B} is the magnetic flux density in the core, \vec{E} is the induced non-conservative electric field, s represents the electric lines, and a is the area of the core. For the coil, the time-varying flux density induces a voltage across its ends, which can be described as

$$V = - nA \frac{d \vec{B}}{dt} \qquad ( 2 )$$

n is the number of turns and A is the cross-sectional area of the core. For the magnetic flux density, one can rewrite \vec{B}=μ_{c}(\vec{H}ₜ+\vec{M}), where μ_{c} is the magnetic permeability inside the coil, \vec{M} is the magnetization of the magnetic wire and \vec{H}ₜ is the total magnetic field which is a sum of ac excitation field \vec{H}ₑ and the external dc magnetic field \vec{H}_{dc}; therefore, the overall field, \vec{H}ₜ=\vec{H}ₑ+\vec{H}_{dc}. Since the magnetic sample is moving through the coil, μ_{c} is also a time-variable.

V=−nA (3)

However, the sample's volume fraction out of the coil is less than 5%, leading to the fact that

$$\frac{d {μ}_{c}}{dt} ( {\vec{H}}_{e} + {\vec{H}}_{d c} + \vec{M} )$$

is negligible. If the sample has low or zero coercivity,

$$\frac{d {\vec{H}}_{d c}}{dt}$$

is negligible, resulting in the reduced form:

$$V = - n A {μ}_{c} ( \frac{d {\vec{H}}_{e}}{dt} + \frac{d \vec{M}}{dt} ) \qquad ( 4 )$$

Using the following expression

$$\frac{d \vec{M}}{dt} = ( \frac{d \vec{M}}{dH} ) \frac{d {\vec{H}}_{e}}{dt}$$

one can rewrite (4) as

$$V = - n A {μ}_{c} ( 1 + \frac{d \vec{M}}{d H} ) \frac{d {\vec{H}}_{e}}{dt} \qquad ( 5 )$$

$$\frac{d \vec{M}}{d H}$$

is the susceptibility of the magnetic wire χ, and 1+χ can be considered as the relative permeability of the magnetic wire μ_{w}.

$$V = - nA {μ}_{c} {μ}_{w} \frac{d {\vec{H}}_{e}}{dt} \qquad ( 6 )$$

with an AC current I and the reactance X=V/I in the coil system, given by X=2πfL, where L is the inductance. Finally, the inductance can be determined using the large length-to-diameter ratio:

$$L \approx \frac{{n}^{2} {μ}_{c} {μ}_{w} A}{l} \qquad ( 7 )$$

where l is the length of the coil. When the magnetic sample is positioned inside the coil, it alters the magnetic permeability of the coil, consequently causing variations in the coil's inductance as the sample travels through it.

A simplified model of a non-ideal coil is considered, consisting of inductance (L) and resistance (R_{L}) in series and parasitic capacitance (C_{L}) in parallel. The complex impedance of the coil (Z_{coil}) has two components in series (R_{L}+iwL) and in parallel to that series

$$( - \frac{i}{{wC}_{L}} ) ,$$

where i is the imaginary unit and w is the angular frequency.

$${Z}_{coil} = \frac{1}{\frac{1}{{R}_{L} + iwL} + \frac{1}{- i / {wC}_{L}}} \qquad ( 8 )$$

Since the magnetic wire is highly conductive, it has very small resistance. As a result, the complex impedance of the coil can be written as

$${Z}_{c o i l} \approx \frac{i w L}{1 - {w}^{2} L {C}_{L}} \qquad ( 9 )$$

At high frequencies, within the capacitive regime, the inductance or reactance becomes negative, and the impedance is inversely proportional to the capacitance. Additionally, in non-ideal coils, high frequency alternating current (AC) induces parasitic c which can be expressed as

$$C = {ε}_{0} {ε}_{r} \frac{N A}{d} \qquad ( 10 )$$

where ε₀ is the permittivity of free space, A is the area of equivalent coil geometry, N is number of turns and d is the separation between them. This means that changes in the dielectric constant εᵣ, which are induced by the sample passing through the sensor, can affect the capacitance (C) and, consequently, the impedance (Z_{coil}). This effect enhances detection sensitivity, even for nanoparticles that lack magnetic properties but possess dielectric characteristics, such as SiO₂. The sensor's detection sensitivity is defined as the change in the total impedance of the system resulting from variations in the magnetic and dielectric signals of the core/shell nanoparticles as they flow through the magnetic coil:

ΔZₜₒₜₐₗ=ΔZₘ+ΔZ_{d} (11)

where ΔZₘ=Zₘ(0)−Zₘ(S) and ΔZ_{d}=Z_{d}(0)−Z_{d}(S), with 0 and S for the coil's impedance without a sample and with a sample, respectively. ΔZₘ and ΔZ_{d} represent the changes in impedance due to the magnetic and dielectric contributions of the core/shell nanoparticles, respectively.

To fabricate the MLCR sensor used in this study, a magnetic coil was constructed from an ultrasoft magnetic Co_{69.25}Fe_{4.25}Si₁₂B_{13.5}Nb₁ microwire (60 μm diameter), wrapped in 15 turns around a plastic tube (diameter: 4 mm; length: 7 mm). This design enables magnetic samples to pass through the coil easily while allowing precise measurement of their impact on the coil's total impedance. For comparison, a conventional GMI sensor, consisting of a single straight microwire with the same Co_{69.25}Fe_{4.25}Si₁₂B_{13.5}Nb₁ composition, was also fabricated.

To ensure accurate impedance measurement, the SubMiniature version A (SMA) port was connected to the magnetic coil or microwire using a soldering gun, establishing a reliable electrical connection. The SMA ports were then linked to a coaxial line and a 50-ohm terminator to match the system's impedance. Following this setup, a wide frequency sweep was performed to assess the sample's impedance characteristics. This sweep was controlled through a LabVIEW program, enabling precise measurement and data acquisition across a broad frequency range, ensuring a comprehensive analysis of the sample's impedance behavior.

A stepper motor was used to control the sample's speed through the sensor. As the sample passed through the sensor's center, its magnetic properties, along with those of the surrounding environment, changed, resulting in a variation in total impedance (ΔZₜₒₜₐₗ). All nanoparticle samples were tested under identical conditions, with a sample speed of approximately 2 cm/s. The observed differences in ΔZₜₒₜₐₗ were attributed to the varying magnetic properties of the samples. The MLCR sensor has been shown to detect both magnetic and dielectric signals from core/shell nanoparticles, such as Fe₃O₄@SiO₂ nanoparticles.

Polycrystalline Fe₃O₄ nanoparticles with varying particle sizes (~190 nm and ~340 nm), each composed of multiple ~9-12 nm nanocrystals, were synthesized using the solvothermal method. A modified Stöber method was then used to coat these Fe₃O₄ nanoparticles with silica to create core/shell nanostructures. The silica shell thickness was adjusted by varying the volume of the Fe₃O₄ dispersion. For ~190 nm cores, 1.25 mL of dispersion was used to achieve a shell thickness of ~30 nm. For ~340 nm cores, 1.25, 2.10, 2.50, and 3.75 mL of dispersion were used to form shells of ~48, ~30, ~25, and ~17 nm, respectively. In each case, the required volume of Fe₃O₄ dispersion was mixed with absolute ethanol (Decon Labs) to reach a total volume of 80 mL in a 250 mL round-bottom flask, followed by the addition of 20 mL of deionized water (18 MΩ·cm, Academic MilliQ-Water system, Millipore Corporation) and sonication for 1.5 h. Afterward, 2.4 mL of ammonium hydroxide solution (28-30%, VWR Chemicals) was introduced and stirred for 10 min at 500 rpm using an overhead stirrer. Then, 183 μL of tetraethyl orthosilicate (99%, Sigma-Aldrich) was added. After stirring for 2 h, the resulting product was collected and washed with ethanol multiple times through magnetic separation and dried overnight in an oven at 60 °C for further analysis.

Based on this, the number of particles between the core-only and core/shell samples was matched: 8.5 mg of core-only particles equate to 14.0 mg of core/shell particles, while 14.0 mg of core-only particles equates to 23.0 mg of core/shell particles. Total mass of 8.5, 11.25, and 14.0 mg was used for the core-only nanoparticles, while the core/shell samples were tested at 8.5, 11.25, 14.0, 18.5, and 23.0 mg.

Additionally, another set of nanoparticles was tested with Fe₃O₄ core diameters of approximately 339 nm, SiO₂ shell thicknesses of 17, 25, 30, and 48 nm, and a crystallite size of 9 nm. This enabled analysis of the effect of shell thickness on changes i while shell masses were calculated as follows: for 17 nm, for 25 nm, for 30 nm, and for 48 nm. For mass equivalence, core-only particle masses of 4.0, 8.0, 11.25, and 14.0 mg were used. For each core/shell particle, corresponding masses were 4.6, 9.8, 13.1, and 16.3 mg for the 17 nm shell; 5, 10.1, 14.2, and 17.6 mg for the 25 nm shell; 5.3, 10.6, 14.9, and 18.5 mg for the 30 nm shell; and 6.3, 12.5, 17.6, and 22.0 mg for the 48 nm shell.

Scanning electron microscopy (SEM) imaging was performed using a FEI Dual Beam 235 Focused Ion Beam system at 15 kV For sample preparation, nanoparticles were dispersed in ethanol and dropped-cast onto clean silicon wafers. Transmission electron microscopy (TEM) analysis was conducted with a JEOL JEM-2010 operating at 200 kV, using 300-mesh holey carbon-coated copper grids (TED Pella). X-ray diffraction (XRD) measurements were taken using a Rigaku Smart Lab system with Cu Kα irradiation at 40 kV and 44 mA, employing a 0.02° step size. Crystallite size was calculated using the Scherrer equation based on the (311) diffraction peak at a 2θ value of 35.5°. Magnetic properties were characterized by a Physical Property Measurement System (PPMS) from Quantum Design, Inc., equipped with a vibrating sample magnetometer. Magnetic measurements were conducted with the range of maximum applied magnetic field of 2 T.

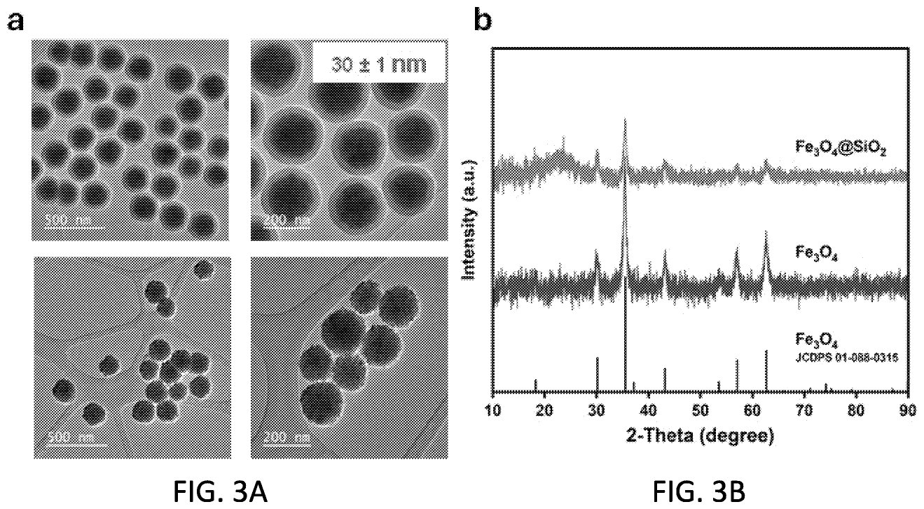

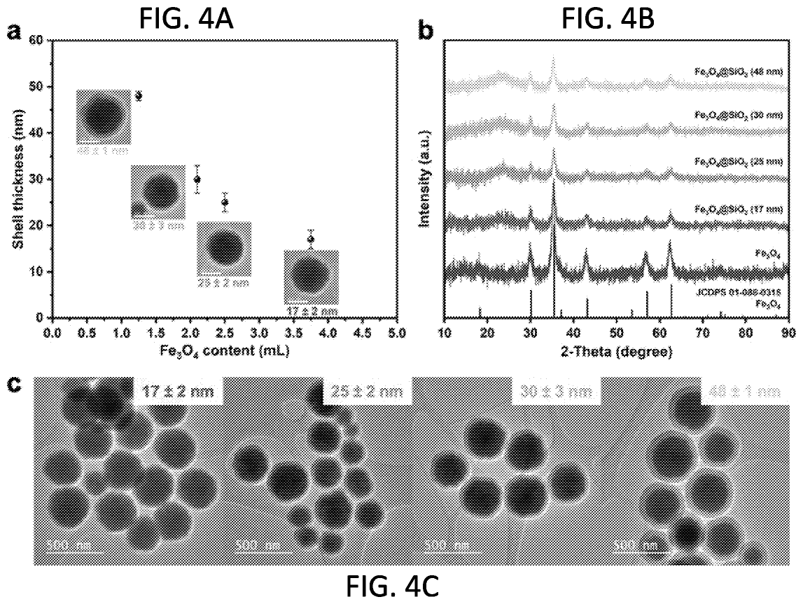

TEM imaging was first performed to assess the structural integrity, interparticle spacing, and crystallite distribution of core Fe₃O₄ and core/shell Fe₃O₄@SiO₂ nanoparticles. The results are presented in FIGS. 3 and 4. FIG. 3 shows the core/shell Fe₃O₄(192 nm)@SiO₂(30 nm) nanoparticles, while FIG. 4 displays Fe₃O₄(339 nm)@SiO₂ nanoparticles with shell thicknesses (tₛ) of 0, 17, 25, 30, and 48 nm. A detailed analysis of the XRD data reveals an average crystallite size (des) (calculated by Scherrer formula) of ~12 nm for Fe₃O₄(192 nm) and ~9 nm for Fe₃O₄(339 nm) nanoparticles, both below the critical size of primary crystals ~15 nm for superparamagnetic behavior. The SiO₂ shell thickness was precisely controlled, ranging from 17 to 48 nm for the core/shell nanoparticles. These sets of samples enabled three sets of data for comparison: (1) the effect of pristine core particle size (192 nm versus 339 nm), (2) the effect of core particle size (192 nm versus 339 nm) with a similar silica shell thickness (~30 nm), and (3) the effect of silica shell thickness (ranging from 0 to 48 nm) with the same core particles size (339 nm), leading to a comprehensive analysis of the data.

XRD characterization further confirmed the phase formation of these nanoparticles. As shown in FIGS. 3 and 4, the XRD patterns exhibit well-defined peaks, indicating good crystallinity. The positions and relative intensities of these peaks correspond to the magnetite (Fe₃O₄) structure, as confirmed by comparison with the Fe₃O₄ JCPDS card (No. 01-088-0315). Notably, when SiO₂ shells were coated on Fe₃O₄ nanoparticles, the primary peaks associated with the Fe₃O₄ phase remained detectable, confirming the retention of the magnetite core. Similar behavior was reported in previous works. The magnetite core, as well as the core/shell nanoparticles, exhibited highly uniform structures with precise control over size and morphology and were used throughout the study. The core/shell nanoparticles displayed a well-defined core/shell structure, with no evidence of core-free particles, multi-core coatings, or aggregations. The quality of the nanoparticles is emphasized as a critical factor for obtaining rational and reliable conclusions in detection applications.

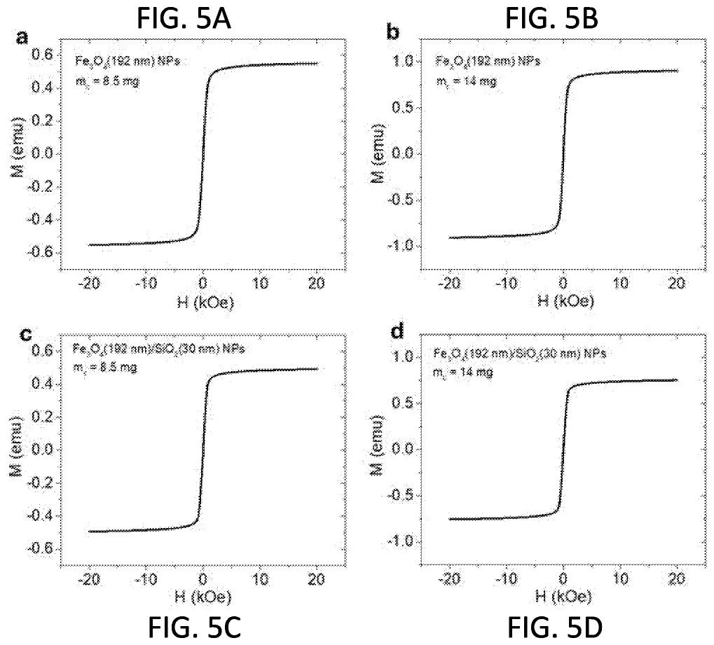

The magnetic properties of the core Fe₃O₄ and core/shell Fe₃O₄@SiO₂ nanoparticles were characterized using the PPMS, with the results presented in FIGS. 5-7. In all cases, the magnetization versus magnetic field (M-H) curves show no magnetic hysteresis, with no coercivity and remanent magnetization (Mᵣ=0, H_{C}=0), confirming the superparamagnetic nature of these nanoparticles. This behavior is expected, as the crystallite size of these nanoparticles (~9-12 nm) is smaller than the critical size (~15 nm) required for exhibiting superparamagnetic behavior in polycrystalline nanoparticles. It has been shown that when the nanoparticle size is below a certain threshold, typically in the range of 10 to 20 nm for many materials like Fe₃O₄, the individual magnetic moments of the particles are no longer able to maintain long-range magnetic order. Instead, they exhibit superparamagnetism, where the nanoparticles behave as if they have a single magnetic domain, and their magnetization can be easily reoriented in response to an external magnetic field. This superparamagnetic behavior is advantageous for biosensing applications, as it allows for easy manipulation and separation of the nanoparticles using an external magnetic field, without the risk of particle aggregation or loss of magnetic responsiveness over time.

The presence of the SiO₂ shell layer reduces the saturation magnetization (Mₛ) in the core/shell Fe₃O₄@SiO₂ nanoparticles compared to the corresponding core Fe₃O₄ nanoparticles, while still retaining the superparamagnetic properties of the core particles. For the same magnetic core mass (m_{c}=8.5 mg), the saturation magnetization decreases from 64.7 emu/g for Fe₃O₄(192 nm) nanoparticles to 57.6 emu/g for core/shell Fe₃O₄(192 nm)@SiO₂(30 nm) nanoparticles (see FIGS. 5a and 5c). When the magnetic core mass increases to 14.0 mg, the saturation magnetization decreases from 64.3 emu/g for Fe₃O₄(192 nm) nanoparticles to 54.3 emu/g for core/shell Fe₃O₄(192 nm)@SiO₂(30 nm) nanoparticles (see FIGS. 5b and 5d).

The consistent saturation magnetization values for the core Fe₃O₄(192 nm) nanoparticles (Mₛ=64.7 emu/g for m_{c}=8.5 mg versus Mₛ=64.3 emu/g for m_{c}=14.0 mg) are expected due to the uniformity in particle size. However, a notable difference in saturation magnetization is observed for the core/shell Fe₃O₄(192 nm)@SiO₂(30 nm) nanoparticles at the two different core masses (Mₛ=57.6 emu/g for m_{c}=8.5 mg versus Mₛ=54.3 emu/g form, =14.0 mg). This reduction in saturation magnetization for these core/shell Fe₃O₄@SiO₂ nanoparticles can be attributed to the increased surface spin disorder at the Fe₃O₄@SiO₂ interface, which is enhanced by the presence of the SiO₂ shell. This spin disordering effect becomes more pronounced in the core/shell structures with larger masses used for the magnetometry measurements.

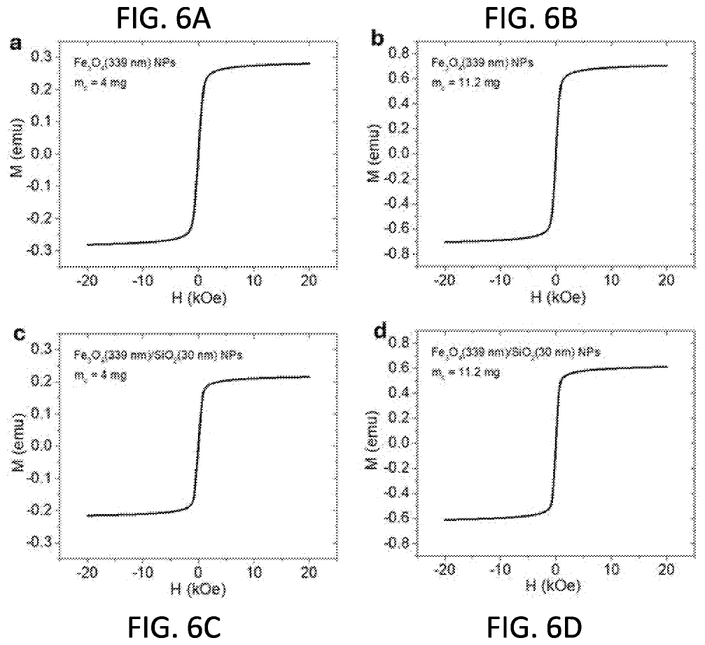

When the Fe₃O₄ core diameter increases from 192 nm to 339 nm, the saturation magnetization increases from 64.7 emu/g for Fe₃O₄(192 nm) nanoparticles to 70.0 emu/g for Fe₃O₄(339 nm) nanoparticles (see FIGS. 5a and 6a). This increase can be attributed to the enhanced crystallinity and improved magnetic properties in the larger nanoparticles. A comparative analysis of the M-H data shows that the saturation magnetization of core/shell Fe₃O₄(192 nm)@SiO₂(30 nm) nanoparticles slightly increases from 54.3 emu/g to 54.46 emu/g for core/shell Fe₃O₄(339 nm)@SiO₂(30 nm) nanoparticles (see FIGS. 5d and 6d). The trend in the change of Mₛ with varying core masses (m_{c}) for core/shell Fe₃O₄(339 nm)@SiO₂(30 nm) nanoparticles (FIG. 6) is similar to that observed above for core/shell Fe₃O₄(192 nm)@SiO₂(30 nm) nanoparticles (FIG. 5). However, the impact of SiO₂ thickness on spin disordering at the Fe₃O₄@SiO₂ interface is less pronounced in the core/shell Fe₃O₄(339 nm)@SiO₂(30 nm) nanoparticles compared to the core/shell Fe₃O₄(192 nm)@SiO₂(30 nm) nanoparticles.

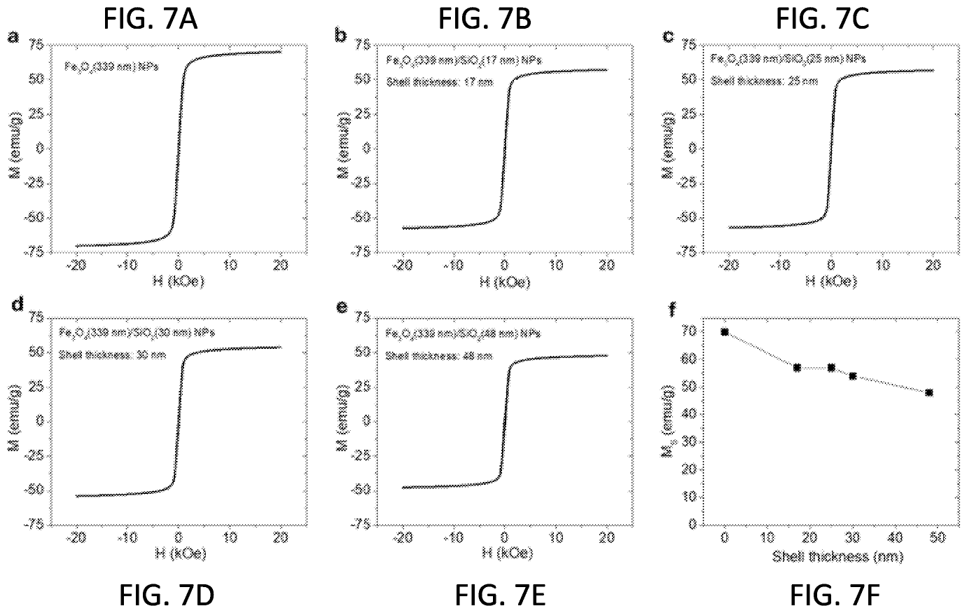

FIG. 7 illustrates how the saturation magnetization of core/shell Fe₃O₄(339 nm)@SiO₂ nanoparticles changes with varying shell thickness, tₛ. While increasing the shell thickness did not alter the superparamagnetic behavior of the core nanoparticles, it did reduce the saturation magnetization. Specifically, increasing the SiO₂ shell thickness from 17 nm to 25 nm had little effect on the saturation magnetization, but further increases in shell thickness led to a significant decrease. This reduction in saturation magnetization is likely due to the enhanced surface spin disordering effect associated with the thicker shell. These changes in saturation magnetization have implications for the magnetic response of the core/shell nanoparticles, particularly in applications that rely on their magnetic properties.

To investigate the effects of magnetic core size, SiO₂ coating, and the variation in shell thickness on the detection sensitivity of the MLCR sensor, a series of biosensing measurements were conducted on the core Fe₃O₄ and core/shell Fe₃O₄@SiO₂ nanoparticles. For comparison, biosensing measurements were also performed on these nanoparticles using the GMI sensor under identical testing conditions. As expected, the GMI sensor detected moderate impedance changes as the nanoparticles flowed through the sensor. Additionally, the impedance change (detection sensitivity) was lower for the core/shell nanoparticles compared to the core nanoparticles. As mentioned earlier, the presence of the non-magnetic SiO₂ shell increased the distance between the GMI sensor and the magnetic cores, leading to a reduction in detection sensitivity. However, the situation is reversed when using the MLCR sensor to detect these nanoparticles.

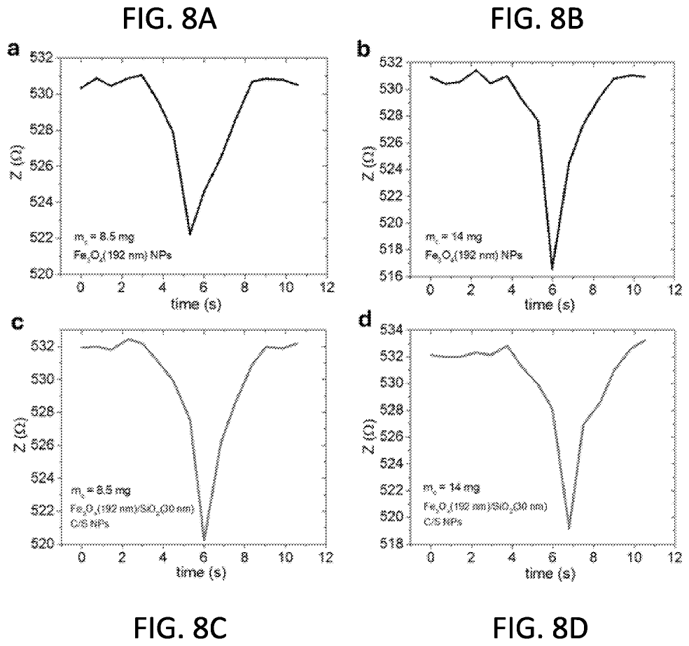

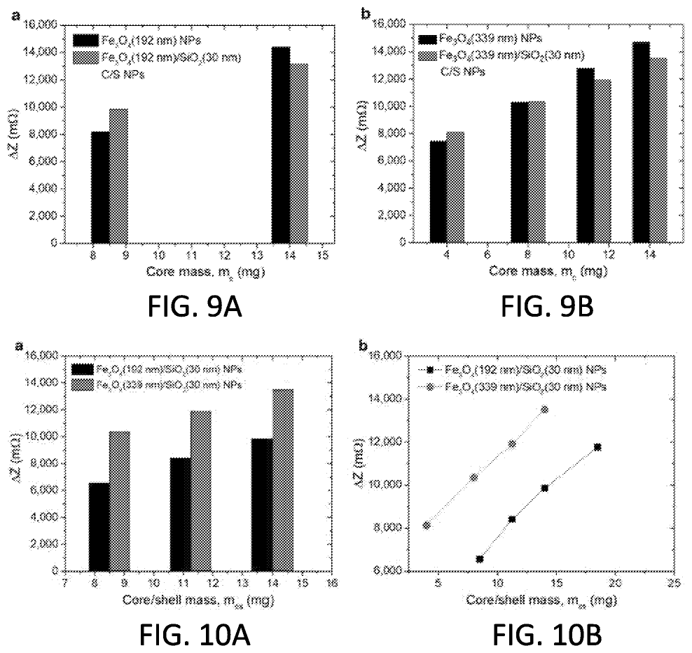

FIG. 8 illustrates the changes in total impedance over time as a sample flowed through the MLCR sensor. It compares core/shell Fe₃O₄(192 nm)@SiO₂(30 nm) nanoparticles with their core-only Fe₃O₄(192 nm) counterparts for two different magnetic core masses, m_{c}=8.5 and 14.0 mg. For the smaller magnetic core mass (m_{c}=8.5 mg), core/shell Fe₃O₄(192 nm)@SiO₂(30 nm) nanoparticles exhibited significantly greater detection sensitivity compared to core Fe₃O₄(192 nm) nanoparticles (see FIGS. 8a and 8c). Specifically, the total impedance change (ΔZ_{coil}) increased from 8,206 mΩ for the core Fe₃O₄(192 nm) nanoparticles to 9,853 mΩ for the core/shell Fe₃O₄(192 nm)@SiO₂(30 nm) nanoparticles, corresponding to an enhancement by ~20%. However, this trend reversed for the larger magnetic core mass (m_{c}=14.0 mg), as shown in FIGS. 8b, 8d, and 9a, where core Fe₃O₄(192 nm) nanoparticles exhibited higher detection sensitivity than their core/shell counterparts.

To confirm these results, the measurements were repeated with another set of samples, using Fe₃O₄ nanoparticles with larger particle sizes (339 nm) and their corresponding core/shell Fe₃O₄(339 nm)@SiO₂(30 nm) nanoparticles. FIG. 9b shows that core/shell Fe₃O₄(339 nm)@SiO₂(30 nm) nanoparticles with smaller magnetic core masses again exhibited superior detection sensitivity compared to their core-only counterparts. Specifically, for m_{c}=4.0 mg, the impedance change (ΔZ_{coil}) increased from 7,446 mΩ for the core Fe₃O₄(339 nm) nanoparticles to 8,120 mΩ for the core/shell Fe₃O₄(339 nm)@SiO₂(30 nm) nanoparticles. However, once the magnetic core mass exceeded a critical value (around 8.5 mg), the detection sensitivity of core Fe₃O₄(339 nm) nanoparticles became dominant over that of the core/shell nanoparticles. These findings suggest that the MLCR sensor is capable of detecting both the magnetic and dielectric signals of core/shell nanoparticles, particularly at lower magnetic core masses, leading to an overall enhancement in detection sensitivity. This ability is particularly important for practical biosensing applications, including disease diagnostics.

A comparative analysis of the detection sensitivity for core/shell Fe₃O₄(192 nm)@SiO₂(30 nm) and Fe₃O₄(339 nm)@SiO₂(30 nm) nanoparticles (see FIG. 10a) reveals that increasing the magnetic core diameter from 192 nm to 339 nm resulted in improved detection sensitivity. This can be attributed to the higher saturation magnetization of the Fe₃O₄(339 nm) cores compared to Fe₃O₄(192 nm) cores. The data indicates a clear correlation between saturation magnetization and detection sensitivity, with both core/shell systems showing a linear increase in sensitivity as a function of magnetic mass (see FIG. 10b), which is advantageous for biosensing applications.

Research has shown that the dielectric constant of bulk SiO₂ typically ranges from 3.9 to 4.0. However, for very thin SiO₂ films, particularly at the nanoscale, the dielectric constant tends to decrease as the thickness reduces. This reduction is due to surface and finite size effects, depolarization effects, and modifications to the atomic structure. Based on this, it is hypothesized that increasing the SiO₂ shell thickness in core/shell nanoparticles, while maintaining the same magnetic cores, can enhance the dielectric constant. This increase in the dielectric constant would contribute to larger impedance changes in the system as these nanoparticles flow through the coil.

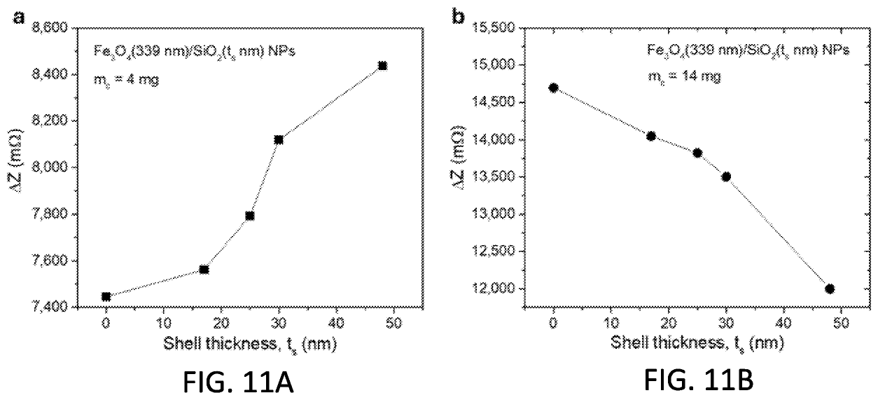

FIG. 11 demonstrates how the total impedance (detection sensitivity) of Fe₃O₄(339 nm)@SiO₂(tₛ nm) nanoparticles changes with varying SiO₂ shell thickness, from 0 to 48 nm, for two different magnetic core masses (m_{c}=4.0 and 14.0 mg). For the smaller core mass (m_{c}=4.0 mg), all core/shell nanoparticles exhibited higher detection sensitivity than the core-only nanoparticles. Interestingly, increasing the SiO₂ shell thickness resulted in a notable increase in detection sensitivity (see FIG. 11a). However, for the larger magnetic core mass (m_{c}=14.0 mg), the opposite effect was observed, with sensitivity decreasing as the shell thickness increased (see FIG. 11b). This trend can be attributed to the dominant contribution of capacitance changes to the total impedance in the case of smaller core masses, due to the enhanced dielectric properties of the SiO₂ shell. In contrast, for larger core masses, the sensor's sensitivity is primarily governed by the magneto-LC resonance effect, which is less influenced by the shell's dielectric properties.

It should also be noted that the presence of the SiO₂ shell can reduce interparticle interactions in core/shell nanoparticles compared to their core-only counterparts. This effect becomes particularly significant in systems with larger magnetic core masses. As the thickness of the SiO₂ shell increases, the distance between neighboring Fe₃O₄ nanoparticles also increases, which reduces their magnetic coupling and diminishes interparticle interactions. These interactions are important for the collective magnetic behavior of the system, and their reduction can affect the overall magnetic properties of the nanoparticles. As a result, the detection sensitivity of the nanoparticles may also be impacted, especially in biosensing applications that rely on strong magnetic interactions for signal detection. For larger magnetic core masses, where the interparticle interactions are stronger, the reduction in these interactions due to the SiO₂ shell may lead to a decrease in the sensor's sensitivity. This underscores the delicate balance between optimizing shell thickness and maintaining sufficient magnetic coupling for enhanced detection performance.

From a biosensing perspective, the above findings are significant because they show that the MLCR sensor is able to detect both magnetic and dielectric signals from core/shell nanoparticles, even at low sample masses. Superior to existing conventional sensors that can only detect either magnetic or dielectric signals of biomarkers, the MLCR sensor's ability to detect subtle dielectric changes is especially valuable for sensing biomaterials that protect magnetic cores in biomedical devices and biosensing systems. In comparison to the GMI sensor for the detection of core/shell nanoparticles, the MLCR sensor exhibits a remarkable enhancement in detection sensitivity, with an increase of approximately 1,653% (see FIG. 9a). These characteristics position the MLCR sensor as a superior tool for magnetic biosensing, offering advantages over traditional sensors like GMR and GMI.

1. A system, comprising:
a coil comprising a soft magnetic microwire having a plurality of turns defining an impedance sensing region; and
a control system comprising:
a coil driver electrically coupled to the coil to deliver an alternating current at a frequency sufficient to induce a negative reactance in the coil;
a transit driver coupled to output a drive signal to cause a sample comprising core/shell nanoparticles to transit through the impedance sensing region;
impedance measurement circuitry electrically coupled to the coil to measure impedance data while the core/shell nanoparticles transit through the impedance sensing region; and
a data output to transmit the impedance data.

2. The system of claim 1, further comprising a sample conduit disposed within the impedance sensing region and configured to carry a sample through the impedance sensing region.

3. The system of claim 2, wherein the sample conduit is a microfluidic cartridge removably receivable in a cartridge receiver of the system.

4. The system of claim 1, wherein the control system further comprises a processor and a non-transitory computer-readable memory storing instructions that, when executed by the processor, cause the processor to:
resolve, from the impedance data, a magnetic impedance component and a dielectric impedance component; and
determine a presence or concentration of a target analyte based on the magnetic impedance component and the dielectric impedance component.

5. The system of claim 4, wherein the instructions further cause the processor to: detect, from the impedance data, a temporal change in the magnetic impedance component or the dielectric impedance component as the core/shell nanoparticles transit through the impedance sensing region; and determine binding of target molecules to the core/shell nanoparticles based on the temporal change.

6. The system of claim 1, further comprising a receptacle storing the core/shell nanoparticles, the core/shell nanoparticles comprising superparamagnetic cores and dielectric shells encapsulating the superparamagnetic cores.

7. The system of claim 6, wherein the dielectric shells are functionalized with a binding moiety to selectively bind a target analyte.

8. The system of claim 6, wherein: the superparamagnetic cores comprise a superparamagnetic oxide having a crystallite size of less than 15 nanometers; the dielectric shells comprise a non-magnetic dielectric material; and a ratio of a diameter of the superparamagnetic cores to a thickness of the dielectric shells is from 4:1 to 30:1.

9. The system of claim 6, wherein the dielectric shells comprise a non-magnetic material having a relative permittivity of at least 2.0 at room temperature.

10. The system of claim 1, further comprising a receptacle storing the core/shell nanoparticles, the core/shell nanoparticles comprising dielectric cores and superparamagnetic shells.

11. The system of claim 1, further comprising a heater configured to deliver energy to the sample within the impedance sensing region while the core/shell nanoparticles transit through the impedance sensing region.

12. The system of claim 11, wherein the control system further comprises a processor and a non-transitory computer-readable memory storing instructions that, when executed by the processor, cause the processor to: resolve, from the impedance data, a magnetic impedance component and a dielectric impedance component; and determine a characteristic of a target analyte based on a change in the dielectric impedance component induced by energy delivered by the heater.

13. A method, comprising: operating a coil driver electrically coupled to a coil to deliver an alternating current at a frequency sufficient to induce a negative reactance in the coil, the coil comprising a soft magnetic microwire having a plurality of turns defining an impedance sensing region; operating a transit driver to output a drive signal to cause a sample comprising core/shell nanoparticles to transit through the impedance sensing region; operating impedance measurement circuitry electrically coupled to the coil to measure impedance data from the core/shell nanoparticles disposed within the sample; and transmitting the impedance data via a data output.

14. The method of claim 13, further comprising providing a sample conduit disposed within the impedance sensing region and carrying the sample through the impedance sensing region via the sample conduit.

15. The method of claim 13, wherein the method further comprises: resolving, from the impedance data, a magnetic impedance component and a dielectric impedance component; and determining a presence or concentration of a target analyte based on the magnetic impedance component and the dielectric impedance component.

16. The method of claim 15, further comprising: detecting, from the impedance data, a temporal change in the magnetic impedance component or the dielectric impedance component as the core/shell nanoparticles transit through the impedance sensing region; and determining binding of target molecules to the core/shell nanoparticles based on the temporal change.

17. The method of claim 13, further comprising operating a heater to deliver energy to the sample within the impedance sensing region while the core/shell nanoparticles transit through the impedance sensing region.

18. The method of claim 17, further comprising: resolving, from the impedance data, a magnetic impedance component and a dielectric impedance component; and determining a characteristic of a target analyte based on a change in the dielectric impedance component induced by energy delivered by the heater.

19. A non-transitory computer-readable medium storing executable instructions to: operate a coil driver electrically coupled to a coil to deliver an alternating current at a frequency sufficient to induce a negative reactance in the coil, the coil comprising a soft magnetic microwire having a plurality of turns defining an impedance sensing region; operate a transit driver to output a drive signal to cause a sample comprising core/shell nanoparticles to transit through the impedance sensing region; operate impedance measurement circuitry electrically coupled to the coil to measure impedance data from the core/shell nanoparticles disposed within the sample; and transmit the impedance data via a data output.

20. The computer-readable medium of claim 19, wherein the instructions are further executable to: resolve, from the impedance data, a magnetic impedance component and a dielectric impedance component; and determine a presence or concentration of a target analyte based on the magnetic impedance component and the dielectric impedance component.
